(19) United States
(12) Patent Application Publication
(10) Pub. No.: US 2003/0108945 A1
(43) Pub. Date: Jun. 12, 2003

(54) Identifying targets of autoreactive T cells in patients with female-prevalent autoimmune diseases and treating these patients

(76) Inventor: Jeffrey Joseph Stewart, Spokane, WA (US)

Correspondence Address: JEFFREY J. STEWART, 2205 E 17th Ave., Spokane, WA, 99203, US

(21) Appl. No.: 09/873,091

(22) Filed: Jun. 1, 2001

Related U.S. Application Data
(63) Continuation of application No. 09/224,894, filed on Jan. 2, 1999, now abandoned.

(51) Int. Cl. G01N 33/53; G01N 33/561

(52) U.S. Cl. 435/7.1; 436/516

(57) ABSTRACT

A method of identifying the primary cellular and molecular targets of female-prevalent autoimmune diseases is presented. The targets are identified by X-chromosome-inactivation patterns of TDC populations. Methods of using these primary cellular and molecular targets as T-cell tolerogens are also presented. Finally, a method is presented for predicting which patients are likely to benefit from treatment methods.

### CROSS REFERENCE TO RELATED APPLICATIONS

This application is a continuation of co-pending application Ser. No. 09/224,894, filed Jan. 2, 1999, which claims the benefit of provisional patent application No. 60/70,575 filed Jan. 6, 1998, both of which are incorporated herein by reference in their entireties.

### BACKGROUND

This invention relates in general to female-prevalent autoimmune diseases such as systemic lupus erythematosus (SLE) and in particular to methods of identifying the primary cellular and molecular targets of autoimmune T cells. This invention also relates to the treatment of the autoimmune condition.

SLE: The Model Female-Prevalent Autoimmune Disease

SLE is an autoimmune disease where tissues and cells are damaged via pathogenic autoantibodies and immune complexes. Clinical manifestations can affect almost any organ system, including the musculoskeletal system, the skin, the central and peripheral nervous systems, the heart, the kidneys, and the gastrointestinal system. The most prevalent symptoms have been used to classify the disease and include malar rash, discoid rash, photosensitivity, oral ulcers, arthritis, serositis, renal dysfunction, neurologic dysfunction, hematologic disorders, immunologic disorders, and the presence of antinuclear antibodies (Hahn, B. H. 1994 In: Harrison's Principles of Internal Medicine pp. 1643-1648).

Females are two to eight times as likely to develop SLE as males, with approximately 1 in 1000 women developing SLE (Hochberg, M. C. 1990 Rheum. Dis. Clin. North Am. 16:617-639; Kotzin, B. L. 1996 Cell 85:303-306). Therefore, in most cases, SLE is generated through a process or processes specific to females. However, it is not known why females are more susceptible to SLE. Hormonal differences have been proposed to alter the course of SLE, but recent studies tracking the effects hormone manipulation can have on the course of the disease have been inconclusive (Asherson, R. A. and R. G. Lahita 1991 Ann. Rheum. Dis. 50:897-898; Buyon, J. P. 1996 Ann. Med. Interne. Paris 147:259-264; Liang, M. H. and E. W. Karlson 1996 Proc. Assoc. Am. Physicians 108:25-28; Jungers, P. et al. 1985 Arthritis Rheum. 28:1243-1250).

The ultimate cause of the disease is unclear. The most widely held theory of the disease's etiology is that the SLE patient's immune system is nonspecifically, polyclonally activated (Klinman, D. M. and A. D. Steinberg 1995 Immunol. Rev. 144:157-193). This has been demonstrated in some female SLE patients (Takeno, M. et al. 1997 J. Immunol. 158:3529-3538). CD4+ T-cells have been isolated from female SLE patients that were able to stimulate autologous B cells to produce both anti-foreign and anti-self antibodies. It has been found that the polyclonal B-cell activation is mediated by B-cell self antigens presented on major histocompatibility complex (MHC) molecules. This observation links T-cell activation to the established correlation of generalized SLE disease activity with polyclonal B-cell activation (Blaese, R. M. et al. 1980 Am. J. Med. 69:345-350). It has been suggested that the autoimmune T cells nonspecifically stimulate B cells, perhaps as a function of the high IL-6 production observed in these T cells (Takeno, M. et al. 1997 J. Immunol. 158:3529-3538).

Overview: X-Inactivation Mosaicism as a Cause of Female-Prevalent Autoimmune Disease

Female-prevalent autoimmune diseases are now thought to be caused by imbalanced X-inactivation chimerism in thymic dendritic cells (TDCs). This imbalance is now believed to lead to a failure of T-cell tolerance (where “tolerance” is the general term for the inactivation or deletion of autoimmune lymphocytes). TDC X-inactivation imbalance is believed to lead to the failure of T-cell tolerance. Females are thought to present to the immune system a self-antigen profile that is radically different from that presented by XY males. This is because females are X-chromosome-inactivation mosaics.

The underlying logic of the proposed etiology for female-prevalent autoimmune disease is as follows: 1) Normally, T cells are screened for autoimmunity in the thymus. 2) The cells that screen the T cells are the TDCs. 3) Autoimmune T cells are those that can interact strongly with the TDCs; if a T cell interacts strongly with a TDC, this autoimmune T cell is tolerized (usually through apoptosis [programmed cell death]). 4) A breakdown in tolerance may occur when there is more than one population of TDCs because T cells that are autoimmune to one but not both populations of the TDCs may escape the tolerance (screening) process. 5) X-chromosome inactivation in females and Klinefelter's males (males with an additional X chromosome) is now believed to cause TDCs to exist in two immunologically distinct populations. 6) Thus, it is believed that female-prevalent autoimmune disease occurs because T cells that are autoimmune to one but not both populations of TDCs fail to interact with that population during the tolerance process, where the populations are defined by the particular X chromosome that is inactivated. 7) When the TDC populations are highly imbalanced, then the T cells that are autoimmune to the minority TDC population are likelier to survive the tolerance process because the appropriate tolerizing cells are infrequent. 8) Thus, high imbalance of the TDC populations is now believed to allow autoimmune T cells to exit the thymus and thus to lead to female-prevalent autoimmune disease.

X-Inactivation Mosaicism

In the late blastocyst stage of early development, each human female cell randomly inactivates one of its X chromosomes, a process that is not mirrored in XY males (Monk, M. J. 1992 J. Inhert. Metab. Dis. 15:499-513; Willard, H. F. 1996 Cell 86:5-7; Lyon, M. F. 1992 Ann. Rev. Genet. 26:16-28; Belmont, J. W. 1996 Am. J. Hum. Genet. 58:1101-1108). The inactivated x chromosome is condensed into a heterochromatin structure (Barr body) that is, for the most part, transcriptionally inactive. The choice of the particular X chromosome that is inactivated in the late blastula stage is faithfully preserved during subsequent cell divisions. Because the clonal descendants of each late blastula cell inactivate the same X chromosome inactivated by the blastula cell, patches of X-chromosome inactivation are created in most tissues. The tortoiseshell coat of the calico cat is a visible example of X-chromosome-inactivation patchiness. In humans, the areas of X-inactivation patchiness in the skin were discovered by Blaschko and are named for him (Blaschko, A. 1901 In: Verhandlungen der Deutschen Dermatolischen Gesellschaft: Siebenter Congress, Neisser, A. (Ed), Wilhelm Braunmuller: Vienna).

X-chromosome inactivation leads females to produce two populations of many cell types that differ in a subset of self antigens (i.e., those antigens expressed and/or presented differentially depending on which X chromosome is active). When these antigenic populations extend to the tolerizing cells of the thymus, some lymphocytes can, it has been proposed, be tolerized by only one of the two self-antigen populations (Kast, R. E. 1977 J. Rheumatol. 4:288-292). Such lymphocytes will be ignorant of the second antigenic population and thus may be autoimmune to self antigens expressed solely by the second antigenic population. The key tolerizing cells are now known to be the TDCs.

Tolerance in the Thymus via TDCS

T-cell maturation, including negative and positive selection, takes place in the thymus (Anderson, G. et al. 1996 Ann. Rev. Immunol. 14:73-99). The thymic cortex is as the primary site of positive selection mediated by epithelial-cell MHC binding to the T-cell receptor (TCR; Busch, R. et al. 1990 Int. Immunol. 2:443-451; Ceppellini, R. et al. 1989 Nature 339:392-394; Bill, J. and E. Palmer 1989 Nature 341:649-651; Berg, L. J. et al. 1989 Cell 58:1035-1046; Benoist, C. and D. Mathis 1989 Cell 58:1027-1033). Negative selection—the main T-cell tolerance process—occurs primarily in the thymic medulla and cortex/medulla junction and is mediated by TDCs (also called interdigitating reticular cells).

TDC-mediated negative selection is potent, with TDCs having been shown to carry out negative selection efficiently even at low titers in culture (van Ewijk, W. et al. 1988 Cell 53:357-370). Studies of murine bone marrow chimeras where MHCs are not present on TDCs demonstrate that one half to one third of all positively selected T cells normally are deleted by interaction with TDCs (van Meerwijk, J. P. et al. 1997 J. Exp. Med. 185:377-383). Related experiments carried out in transgenic mice expressing MHCs in the thymic cortex but not medulla demonstrate that at least 1 out of 20 positively selected T cells are autoimmune to syngeneic splenocytes and are therefore autoimmune if the TDC barrier is compromised (Laufer, T. M. et al. 1996 Nature 383:81-85).

Examination of the human thymus suggests that if a T cell were to follow a straight line from the cortex through the medulla, the T cell would contact a limited number of TDCs, i.e., fewer than 15 (Stewart, J. J. 1998 Immunol. Today 19:352-357). The generally non-autoimmune status of most humans implies that this narrow TDC barrier is sufficient to prevent most autoimmunity.

X-Inactivation Mosaicism in the Thymus

Because X chromosomes are inactivated randomly at the late blastula stage, it was previously believed each “X-population” (a subpopulation of cells defined by the particular X chromosome that is inactivated), including those of the hematopoietic stem cells (those cells that generate dendritic cells, T cells, and B cells), in females approached a 50:50 ratio (i.e., balanced). However, severe imbalance has been shown in many females. In normal females, T-cell X-population imbalances as high as 14:86 (Puck, J. M. et al. 1992 Am. J. Hum. Genet. 50:742-748) and 10:90 (Naumova, A. K. et al. 1996 Am. J. Hum. Genet. 58:1111-1119) have been reported. Puck et al. (1992 Am. J. Hum. Genet. 50:742-748) calculate from the binomial theorem and the observed T-cell X-population imbalance that an extremely small number of late blastula cells eventually give rise to the entire totipotent hematopoietic stem-cell population (6 to 19, with 10 as the maximum likelihood number).

Negative selection mediated by thymic epithelial cells (TECs) appear to be incapable of rescuing many highly-imbalanced immune systems from thymus inefficiency because most if not all TECs arise from a single late blastula cell, so all the TECs have inactivated the same X chromosome (Kendall, M. D. et al. 1988. Cell Tissue Res. 254:283-294; Wilcox, N. et al. 1987. Am. J. Pathol. 127:447-460). TECs thus have an insignificant effect on females whose TDC population is highly imbalanced toward the same X-inactivation type. In other cases, TECs may present an MHC antigen profile that does not include one or more of the X-Ags presented by the TDCs. In these cases, then the TECs will be unable to tolerize the T cells (Bonomo, A. and P. Matzinger 1993 J. Exp. Med. 177:1153-1164).

Accordingly, it is now believed that the polyreactive T cells isolated from SLE patients by Takeno et al. (Takeno, M. et al. 1997 J. Immunol. 158:3529-3538) are each specifically reactive against a particular B-cell self antigen, namely against one of the X-Ags (Stewart, J. J. 1998 Immunol. Today 19:352-357). The high level of IL-6 production in the SLE T cells is now believed to be a result of the autoimmunity rather than the cause of the autoimmunity, as proposed previously by Takeno et al. The imbalance of the TDC X-populations described herein also accounts for the finding of Takeno et al. that autoimmune T cells are able to stimulate and be stimulated by autologous B cells, while there is no similar reaction with MHC-matched B cells from another patient, namely, that the X-Ags are now believed to differ between the patients. Finally, the imbalance is now believed to explain why the autoimmune T cells' TCRs do not themselves recognize heat shock protein 60 or contain the anionic motifs often seen in anti-DNA antibodies (Takeno, M. et al. 1997 J. Immunol. 158:3529-3538; Stewart, J. J. 1998 Immunol. Today 19:352-357).

The Present Invention

Whatever the precise merits, features, and advantages of the above cited prior art teachings, none of them achieves or fulfills the purpose of determining the primary cellular and molecular targets of autoimmune T cells in female-prevalent autoimmune disease.

An object of the present invention is a method of identifying, in a manner more specific than previously

1. Isolating the patient's TDCs. Methods of isolating TDCs are well known to those with skill in the art (see, for example, Fearnley, D. B. et al. 1997 Blood 15:3708-3716).

2. Determining the minority and majority X-populations of the TDCs. Methods of determining the minority and majority X-populations are well known to those with skill in the art (see, for example, Naumova, A. K. et al. 1996 Am. J. Hum. Genet. 58:1111-1119; Fey, M. F. et al. 1992 Blood 83:931-8; and Vogelstein, B. et al. 1987 Cancer Res. 47: 4806-13).

Peripheral TDCS

In all the aforementioned inventions and embodiments thereof, the preferred embodiment of determining the minority and majority X-populations of TDCs comprises determining the dendritic-cell X-populations found in the periphery (it is currently believed that the thymic and peripheral dendritic cell X-populations exist at equivalent ratios).

General

All publications cited in this specification are herein incorporated by reference as if each individual publication were specifically and individually indicated to be incorporated by reference herein as though fully set forth.

In general, the methods of the invention may be alternatively composed to comprise, consist of, or consist essentially of any appropriate method herein disclosed, and such compositions of the invention may additionally, or alternatively, be formulated so as to be devoid, or substantially free, of any components, methods, materials, ingredients, adjuvants, or species used in prior art compositions or that otherwise are not necessary to the achievement of the function and objectives of the present invention.

The foregoing descriptions of the preferred embodiments of the invention have been presented for the purposes of illustration and description. They are not intended to be exhaustive or to limit the invention to the precise forms disclosed. Many modifications and variations are possible in the light of the above teachings. It is intended that the scope of the invention be limited not by the detailed description but rather by the claims appended hereto.

### EXAMPLES

The following examples are presented for illustration purposes. It is not intended that the following examples limit the scope of the invention in any way.

### Example 1

### Turner's Syndrome

Turner's Syndrome patients provide an example where X-chromosome inactivation chimerism appears to induce autoimmune disease. Approximately 50% of females diagnosed with Turner's syndrome have been shown to be chromosomal mosaics. These patients often have not only XO cells but also cells containing abnormal sex chromosomes, including, X isochromosomes, XX, XY, XXX, XYY, and ring X, and Y chromosomes (Jacobs, P. A. et al. 1990 Ann. Hum. Genet. 54:209-223; Magenis, R. E. et al. 1980 Am. J. Hum. Genet. 32:79A). Furthermore, X isochromosomes initiate lymphocyte X-inactivation imbalance, presumably because isochromosomes are preferentially inactivated (Zinman, B. et al. 1984. Clin. Invest. Med. 7:135-141). Accordingly, a large number of patients with Turner's syndrome suffer from autoimmune diseases (Larizza, D. et al. 1989 Autoimmunity 4:69-78; Chivato, L. et al. 1996 Eur. J. Endocrinol. 134:568-575; Balestrazzi, P. et al. 1986 Clin. Exp. Rheumatol. 4:61-62; Marcocci, C. et al. 1980 J. Endocrinol. Invest. 3:429-431; Chen, H. et al. 1978 J. Birth Defects 14:137-147; Sparkes, R. S. and A. G. Motulsky 1967 Ann. Intern. Med. 67:132-144). The minority X-population of TDCs in each patient is identified as the cellular target of autoimmunity.

### Example 2

### X-Linked Dyskeratosis Congenita Carriers

Carriers of X-linked dyskeratosis congenita exhibit a high X-inactivation imbalance. These subjects exhibit an abnormally high incidence of autoimmune disease and aberrant T-cell function (Ling, N. S. et al. 1985 Arch. Dermatol. 121:1424-1428; Fudenberg, H. H. et al. 1979 Gerontology 25:231-237). The minority X-population of TDCs in each patient is identified as the cellular target of autoimmunity.

### Example 3

### Klinefelter's Syndrome

Males with Klinefelter's syndrome exhibit X-inactivation chimerism. Unlike normal, XY males, Klinefelter's males have two X chromosomes in addition to the Y chromosome. Accordingly, Klinefelter's syndrome correlates with an increased prevalence of autoimmune disease, including SLE (Merchant, P. C. and S. M. Shahani 1989 Andrologia 21:476-478; Schattner, A. and A. Berrebi 1989 J. Royal Soc. Med. 82:560; Landwirth, J. and A. Berger 1973 Am. J. Dis. Child. 126:851-853; Ortiz-Neu, C. and E. C. LeRoy 1969 Arthritis Rheum. 12:241-246; Burch, P. R. et al. 1966 Lancet 2:748-749; Stern, R. et al. 1977 Arthritis Rheum. 20:18-22). The minority X-population of TDCs in each patient is identified as the cellular target of autoimmunity.

### Example 4

### Autoimmune Blaschkitis

Autoimmune symptoms may preferentially present along Blaschko lines. In these examples, autoimmune attack of cells in one Blaschko line with little or no attack in adjacent lines indicates that X-populations preset relevant antigenic targets for autoimmune attack. Autoimmune diseases presenting visible symptoms along Blaschko lines include lichenoid eruptions (Breathnach, S. M. and S. I. Katz 1986 Hum. Pathol. 17:161-167) and linear scleroderma (Kleiner-Baumgarten, A. et al. 1989 J. Rheumatol. 16:1141-1143) follow the lines of Blaschko (Taieb, A. et al. 1991 J. Am. Acad. Dermatol. 25:637-642; Kennedy, D. and M. Rogers 1996 Pediatr. Dermatol. 13:95-99). The minority X-population of TDCs in each patient is identified as the cellular target of autoimmunity.

### Example 5

### TDC X-Inactivation

The small number of hematopoietic stem cells that generate the TDCs indicates that highly imbalanced TDC populations are rare but not absent from the human female population (Puck, J. M. et al. 1992 Am. J. Hum. Genet. 50:742-748). For example, 10:90 lymphocyte imbalance will occur stochastically in roughly 2% of females if 10 late blastula cells are the progenitors for the hematopoietic stem-cell population (Stewart, J. J. 1998 Immunol. Today 19:352-357). The minority X-population of TDCs in each patient is identified as the cellular target of autoimmunity.

### Example 6

### Tolerance Escape in the X-Imbalanced Thymus

The more extreme the X-inactivation imbalance of TDCs, the greater the likelihood that autoimmune T cells will pass through the negative-selection gauntlet. The expected ratio of T cells that escape negative selection as a function of the TDC X-population imbalance and of the number of TDCs that have the opportunity to tolerize each T cell is calculated as follows. The probability, p, that a T cell is not tolerized by a particular TDC X-population is given by the formula p=(1−s)ⁿ, where s is the fraction of TDCs of a particular X-population and n is the number of TDCs that screen each T cell. To calculate the overall probability of tolerance escape, the individual probabilities of escaping tolerance from each TDC X-population are summed, i.e., (1−S)ⁿ+sⁿ, (Stewart, J. J. 1998 Immunol. Today 19:352-357).

When the TDC X-populations are balanced, and if 15 TDCs screen each T cell, the fraction of non-tolerized T cells is less than 1 in 10,000; however, if the same 15 TDCs are 10:90 imbalanced, as is expected in 2% of the female population, 20% of T cells will avoid tolerance by one TDC X-population (Stewart, J. J. 1998 Immunol. Today 19:352-357).

SLE: The Model Female-Prevalent Autoimmune Disease

SLE is an autoimmune disease where tissues and cells are damaged via pathogenic autoantibodies and immune complexes. Clinical manifestations can affect almost any organ system, including the musculoskeletal system, the skin, the central and peripheral nervous systems, the heart, the kidneys, and the gastrointestinal system. The most prevalent symptoms have been used to classify the disease and include malar rash, discoid rash, photosensitivity, oral ulcers, arthritis, serositis, renal dysfunction, neurologic dysfunction, hematologic disorders, immunologic disorders, and the presence of antinuclear antibodies (Hahn, B. H. 1994 In: Harrison's Principles of Internal Medicine pp. 1643-1648).

Females are two to eight times as likely to develop SLE as males, with approximately 1 in 1000 women developing SLE (Hochberg, M. C. 1990 Rheum. Dis. Clin. North Am. 16:617-639; Kotzin, B. L. 1996 Cell 85:303-306). Therefore, in most cases, SLE is generated through a process or processes specific to females. However, it is not known why females are more susceptible to SLE. Hormonal differences have been proposed to alter the course of SLE, but recent studies tracking the effects hormone manipulation can have on the course of the disease have been inconclusive (Asherson, R. A. and R. G. Lahita 1991 Ann. Rheum. Dis. 50:897-898; Buyon, J. P. 1996 Ann. Med. Interne. Paris 147:259-264; Liang, M. H. and E. W. Karlson 1996 Proc. Assoc. Am. Physicians 108:25-28; Jungers, P. et al. 1985 Arthritis Rheum. 28:1243-1250).

The ultimate cause of the disease is unclear. The most widely held theory of the disease's etiology is that the SLE patient's immune system is nonspecifically, polyclonally activated (Klinman, D. M. and A. D. Steinberg 1995 Immunol. Rev. 144:157-193). This has been demonstrated in some female SLE patients (Takeno, M. et al. 1997 J. Immunol. 158:3529-3538). CD4+ T-cells have been isolated from female SLE patients that were able to stimulate autologous B cells to produce both anti-foreign and anti-self antibodies. It has been found that the polyclonal B-cell activation is mediated by B-cell self antigens presented on major histocompatibility complex (MHC) molecules. This observation links T-cell activation to the established correlation of generalized SLE disease activity with polyclonal B-cell activation (Blaese, R. M. et al. 1980 Am. J. Med. 69:345-350). It has been suggested that the autoimmune T cells nonspecifically stimulate B cells, perhaps as a function of the high IL-6 production observed in these T cells (Takeno, M. et al. 1997 J. Immunol. 158:3529-3538). females and Klinefelter's males (males with an additional X chromosome) is now believed to cause TDCs to exist in two immunologically distinct populations. 6) Thus, it is believed that female-prevalent autoimmune disease occurs because T cells that are autoimmune to one but not both populations of TDCs fail to interact with that population during the tolerance process, where the populations are defined by the particular X chromosome that is inactivated. 7) When the TDC populations are highly imbalanced, then the T cells that are autoimmune to the minority TDC population are likelier to survive the tolerance process because the appropriate tolerizing cells are infrequent. 8) Thus, high imbalance of the TDC populations is now believed to allow autoimmune T cells to exit the thymus and thus to lead to female-prevalent autoimmune disease.

X-Inactivation Mosaicism

In the late blastocyst stage of early development, each human female cell randomly inactivates one of its X chromosomes, a process that is not mirrored in XY males (Monk, M. J. 1992 J. Inhert. Metab. Dis. 15:499-513; Willard, H. F. 1996 Cell 86:5-7; Lyon, M. F. 1992 Ann. Rev. Genet. 26:16-28; Belmont, J. W. 1996 Am. J. Hum. Genet. 58:1101-1108). The inactivated X chromosome is condensed into a heterochromatin structure (Barr body) that is, for the most part, transcriptionally inactive. The choice of the particular X chromosome that is inactivated in the late blastula stage is faithfully preserved during subsequent cell divisions. Because the clonal descendants of each late blastula cell inactivate the same X chromosome inactivated by the blastula cell, patches of X-chromosome inactivation are created in most tissues. The tortoiseshell coat of the calico cat is a visible example of X-chromosome-inactivation patchiness. In humans, the areas of X-inactivation patchiness in the skin were discovered by Blaschko and are named for him (Blaschko, A. 1901 In: Verhandlungen der Deutschen Dermatolischen Gesellschaft: Siebenter Congress, Neisser, A. (Ed), Wilhelm Braunmuller: Vienna).

X-chromosome inactivation leads females to produce two populations of many cell types that differ in a subset of self antigens (i.e., those antigens expressed and/or presented differentially depending on which X chromosome is active). When these antigenic populations extend to the tolerizing cells of the thymus, some lymphocytes can, it has been proposed, be tolerized by only one of the two self-antigen populations (Kast, R. E. 1977 J. Rheumatol. 4:288-292). Such lymphocytes will be ignorant of the second antigenic population and thus may be autoimmune to self antigens expressed solely by the second antigenic antigen, namely against one of the X-Ags (Stewart, J. J. 1998 Immunol. Today 19:352-357). The high level of IL-6 production in the SLE T cells is now believed to be a result of the autoimmunity rather than the cause of the autoimmunity, as proposed previously by Takeno et al. The imbalance of the TDC X-populations described herein also accounts for the finding of Takeno et al. that autoimmune T cells are able to stimulate and be stimulated by autologous B cells, while there is no similar reaction with MHC-matched B cells from another patient, namely, that the X-Ags are now believed to differ between the patients. Finally, the imbalance is now believed to explain why the autoimmune T cells' TCRs do not themselves recognize heat shock protein 60 or contain the anionic motifs often seen in anti-DNA antibodies (Takeno, M. et al. 1997 J. Immunol. 158:3529-3538; Stewart, J. J. 1998 Immunol. Today 19:352-357).

The Present Invention

Whatever the precise merits, features, and advantages of the above cited prior art teachings, none of them achieves or fulfills the purpose of determining the primary cellular and molecular targets of autoimmune T cells in female-prevalent autoimmune disease.

An object of the present invention is a method of identifying, in a manner more specific than previously

1. Isolating the patient's TDCs. Methods of isolating TDCs are well known to those with skill in the art (see, for example, Fearnley, D. B. et al. 1997 Blood 15:3708-3716).

2. Determining the minority and majority X-populations of the TDCs. Methods of determining the minority and majority X-populations are well known to those with skill in the art (see, for example, Naumova, A. K. et al. 1996 Am. J. Hum. Genet. 58:1111-1119; Fey, M. F. et al. 1992 Blood 83:931-8; and Vogelstein, B. et al. 1987 Cancer Res. 47: 4806-13).

Peripheral TDCS

In all the aforementioned inventions and embodiments thereof, the preferred embodiment of determining the minority and majority X-populations of TDCs comprises determining the dendritic-cell X-populations found in the periphery (it is currently believed that the thymic and peripheral dendritic cell X-populations exist at equivalent ratios).

General

All publications cited in this specification are herein incorporated by reference as if each individual publication were specifically and individually indicated to be incorporated by reference herein as though fully set forth.

In general, the methods of the invention may be alternatively composed to comprise, consist of, or consist essentially of any appropriate method herein disclosed, and such compositions of the invention may additionally, or alternatively, be formulated so as to be devoid, or substantially free, of any components, methods, materials, ingredients, adjuvants, or species used in prior art compositions or that otherwise are not necessary to the achievement of the function and objectives of the present invention.

The foregoing descriptions of the preferred embodiments of the invention have been presented for the purposes of illustration and description. They are not intended to be exhaustive or to limit the invention to the precise forms disclosed. Many modifications and variations are possible in the light of the above teachings. It is intended that the scope of the invention be limited not by the detailed description but rather by the claims appended hereto.

### EXAMPLES

The following examples are presented for illustration purposes. It is not intended that the following examples limit the scope of the invention in any way.

### Example 2

### X-Linked Dyskeratosis Congenita Carriers

Carriers of X-linked dyskeratosis congenita exhibit a high X-inactivation imbalance. These subjects exhibit an abnormally high incidence of autoimmune disease and aberrant T-cell function (Ling, N. S. et al. 1985 Arch. Dermatol. 121:1424-1428; Fudenberg, H. H. et al. 1979 Gerontology 25:231-237). The minority X-population of TDCs in each patient is identified as the cellular target of autoimmunity.

### Example 3

### Klinefelter's Syndrome

Males with Klinefelter's syndrome exhibit X-inactivation chimerism. Unlike normal, XY males, Klinefelter's males have two X chromosomes in addition to the Y chromosome. Accordingly, Klinefelter's syndrome correlates with an increased prevalence of autoimmune disease, including SLE (Merchant, P. C. and S. M. Shahani 1989 Andrologia 21:476-478; Schattner, A. and A. Berrebi 1989 J. Royal Soc. Med. 82:560; Landwirth, J. and A. Berger 1973 Am. J. Dis. Child. 126:851-853; Ortiz-Neu, C. and E. C. LeRoy 1969 Arthritis Rheum. 12:241-246; Burch, P. R. et al. 1966 Lancet 2:748-749; Stern, R. et al. 1977 Arthritis Rheum. 20:18-22). The minority X-population of TDCs in each patient is identified as the cellular target of autoimmunity. stem-cell population (Stewart, J. J. 1998 Immunol. Today 19:352-357). The minority X-population of TDCs in each patient is identified as the cellular target of autoimmunity.

### Example 6

### Tolerance Escape in the X-Imbalanced Thymus

The more extreme the X-inactivation imbalance of TDCs, the greater the likelihood that autoimmune T cells will pass through the negative-selection gauntlet. The expected ratio of T cells that escape negative selection as a function of the TDC X-population imbalance and of the number of TDCs that have the opportunity to tolerize each T cell is calculated as follows. The probability, p, that a T cell is not tolerized by a particular TDC X-population is given by the formula p=(1−s)ⁿ, where s is the fraction of TDCs of a particular X-population and n is the number of TDCs that screen each T cell. To calculate the overall probability of tolerance escape, the individual probabilities of escaping tolerance from each TDC X-population are summed, i.e., (1−S)ⁿ+Sⁿ (Stewart, J. J. 1998 Immunol. Today 19:352-357).

When the tdc x-populations are balanced, and if 15 TDCs screen each t cell, the fraction of non-tolerized T cells is less than 1 in 10,000; however, if the same 15 TDCs are 10:90 imbalanced, as is expected in 2% of the female

I claim:

1. A method of identifying a target of autoimmunity in a patient suffering from a female-prevalent autoimmune disease, said method comprising:
isolating said patient's TDCs;
determining the minority and majority X-populations of said TDCs; and
identifying said minority X-population as the cellular target of autoimmunity.

2. A method of claim 1 where the female-prevalent autoimmune disease is selected from the group consisting of SLE, myasthenia gravis, Sjogren's syndrome, Hashimoto's thyroiditis, Grave's disease, primary biliary cirrhosis, chronic active hepatitis, chronic idiopathic thrombocytopenic purpura, polymyositis, dermatomyositis, polymyalgia rheumatica, idiopathic Addison's disease, progressive systemic sclerosis, and rheumatoid arthritis.

3. A method of claim 1 where said patient is a human female or human Klinefelter's male.

4. A method of claim 1 where said minority and majority X-populations of TDCs are determined by determining the dendritic-cell X-populations found in said patient's periphery.

5. A method of claim 1 further comprising the steps of:
isolating MHC-associated peptides from said minority X-population;
isolating MHC-associated peptides from said majority X-population;
comparing said minority peptides with said majority peptides; and
identifying peptides more commonly isolated from the minority X-population than from the majority X-population as the molecular targets of autoimmunity.

6. A method of claim 1 further comprising the steps of:
preparing two-dimensional gels from said majority and minority X-populations;
identifying proteins or mRNAs on said gels;
comparing proteins or mRNAs on said gels;
identifying proteins or mRNAs on said gels that are more highly observed on said minority TDC X-population than on said majority TDC X-population as the molecular target precursor;
determining T-cell epitopes of the proteins comprising said molecular target precursor or of the proteins specified by translation of the mRNA comprising said molecular target precursor; and
identifying said T-cell epitopes as the molecular targets of autoimmunity.

7. A method of tolerizing autoreactive T-cells of a patient suffering from a female-prevalent autoimmune disease, said method comprising:
identifying an X-population target of autoimmunity;
preparing said target to tolerize T cells; and
contacting said target with said T cells.

8. A method of claim 7 wherein said target is a cellular target, where said cellular target is identified by a method comprising:
isolating said patient's TDCs;
determining the minority and majority X-populations of said TDCs;
identifying said minority X-population as the cellular target of autoimmunity;

9. A method of claim 7 wherein said target is a molecular target, where said molecular target is identified by a method comprising:
isolating said patient's TDCs;
determining said minority and majority X-populations of said TDCs;
isolating MHC-associated peptides from said majority TDC X-population;
isolating MHC-associated peptides from said minority DC X-population;
comparing said minority peptides to said majority peptides; and
identifying peptides more commonly isolated from the minority X-population than from the majority X-population as the molecular targets of autoimmunity.

10. A method of claim 7 wherein said target is a molecular target, where said molecular target is identified by a method comprising:
isolating said patient's TDCs;
determining said minority and majority X-populations of said TDCs;
preparing a two-dimensional gel from said minority X-population;
preparing a two-dimensional gel from said majority X-population;
comparing said gels;
identifying on said gels dimorphic proteins or mRNAs that are specific to or are more highly expressed by the minority TDC X-population relative to the majority TDC X-population as the molecular target precursor;
determining T-cell epitopes of said molecular target precursor;
identifying said epitopes as molecular targets of autoimmunity.

11. A method of predicting patients likely to benefit from a treatment directed to ameliorating the effects of X-population imbalance, said method comprising:
isolating said patient's TDCs;
determining the minority and majority X-populations of said TDCs; and
identifying patients whose said majority X-population is significantly larger than said minority X-population as patients likely to benefit from said treatment.
